(19) United States
(12) Patent Application Publication
(10) Pub. No.: US 2008/0207945 A1
(43) Pub. Date: Aug. 28, 2008

(54) Preparation of gabapentin by liquid-liquid extraction

(76) Inventors: Ziv Dee-Noor, Haifa (IL); Amir Gold, Herzelia (IL)

Correspondence Address: KENYON & KENYON LLP, ONE BROADWAY, NEW YORK, NY, 10004, US

(21) Appl. No.: 12/072,779

(22) Filed: Feb. 28, 2008

Related U.S. Application Data
(60) Provisional application No. 60/904,211, filed on Feb. 28, 2007.
(60) Provisional application No. 60/921,668, filed on Apr. 2, 2007.
(60) Provisional application No. 60/927,633, filed on May 3, 2007.
(60) Provisional application No. 60/930,190, filed on May 14, 2007.

(51) Int. Cl. C07C 227/16

(52) U.S. Cl. 562/507

(57) ABSTRACT

This invention relates to an efficient process for converting gabapentin hydrochloride salt to gabapentin by liquid-liquid extraction using a counter-current extraction method.

### CROSS-REFERENCE TO RELATED APPLICATIONS

This application claims the benefit of U.S. provisional application Ser. Nos. 60/904,211, filed Feb. 28, 2007; 60/921,668, filed Apr. 2, 2007; 60/927,633, filed May 3, 2007; and 60/930,190, filed May 14, 2007, hereby incorporated by reference.

### FIELD OF THE INVENTION

This invention relates to an efficient process for converting gabapentin hydrochloride salt to gabapentin.

### BACKGROUND OF THE INVENTION

Gabapentin (referred to as “GBP”), 1-(aminomethyl)cyclohexaneacetic acid has a molecular formula of C₉ H₁₇NO₂ and a molecular weight of 171.24. The structure of gabapentin is:

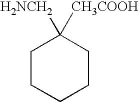

GBP is a white to off-white crystalline solid with a pKₐ₁ of 3.7 and a pKₐ₂ of 10.7. GBP is marketed by Pfizer under the trade name Neurontin®. The following dosage forms of Neurontine are available: capsules, tablets, and oral solution are supplied in several strengths. The commercially available gabapentin is crystalline and hereinafter, the commercially available polymorphic form of gabapentin is referred to as form II.

GBP is used in the treatment of cerebral diseases such as epilepsy. In animal models of analgesia, gabapentin prevents allodynia (pain-related behavior in response to a normally innocuous stimulus) and hyperalgesia (exaggerated response to painful stimuli). Gabapentin also decreases pain related responses after peripheral inflammation. Animal test systems designed to detect anticonvulsant activity, proved that gabapentin prevents seizures as do other marketed anticonvulsants.

U.S. Pat. No. 4,024,175 describes several methods of preparing gabapentin from cyclohexyl-1,1-diacetic acid. Each of these methods results in the formation of gabapentin hydrochloride salt (referred to as “GBP-HCl”), which is converted to GBP by treatment with a basic ion exchanger, and then crystallized from a solvent mixture such as acetone/ethanol/ether.

U.S. Pat. No. 4,894,476 refers to a method for converting the hydrochloride salt into a crystalline gabapentin monohydrate by eluting an aqueous solution that contains GBP-HCl through a basic ion-exchange resin, producing a slurry from the eluate, adding an alcohol to the slurry and isolating the final product.

WO 2004/110342 describes the formation of a crystalline form of gabapentin, characterized by the following PXRD peaks: 6.3, 12.6, 16.3, 18.0, 18.8, 19.4, 21.4, 25.3, 26.3, 27.0, 30.2, 32.4, 35.7, 38.2 and 45.6±0.2 degrees theta, referred to as gabapentin form IV by neutralizing gabapentin salt with a base at a specified temperature range in different solvents. Gabapentin form IV is then converted to give a crystalline form of gabapentin characterized by the following PXRD peaks: 7.8, 13.3, 14.9, 16.6, 16.8, 19.5, 20.2, 21.3, 21.8, 23, 23.5, 25.7, 26.9 and 28±0.2 degrees theta, referred to as gabapentin form II.

WO 2004/093779 describes a process for the preparation of gabapentin by neutralizing gabapentin hydrochloride solution with a base at high temperatures, followed by cooling to yield a crystalline Form II of gabapentin.

WO 2004/110981 describes a process for preparing gabapentin form II by reacting 1,1-cyclohexane diacetic acid mono amide with alkali hypohalite, followed by acidification in the presence of organic solvents, extracting the obtained salts, and adding an ante solvent to crystallize gabapentin acid salts. The intermediate salts are then isolated, suspended in an organic solvent under basic conditions at a specified temperature range to obtain gabapentin form III which is then converted to gabapentin form II.

U.S. Pat. No. 6,255,526 relates to a method for converting gabapentin hydrochloride substantially free of inorganic salts to gabapentin form II.

The above methods require the use of large amounts of organic solvents and various pH ranges. Also, further purification steps such as isolation and drying gabapentin HCl are required. The above mentioned processes often require many steps and are time consuming and complicated, and therefore are expensive, especially when adapting these processes for industrial scale operations.

Hence, there is a need in the art for a more efficient, convenient and industrially applicable process for converting GBP-HCl to GBP.

### SUMMARY OF THE INVENTION

In one embodiment, the invention encompasses a method for converting GBP-HCl to gabapentin comprising: extracting GBP-HCl from an aqueous mixture with an extraction solvent having at least one C₄-C₇ alcohol; and reacting the extracted GBP-HCl with a base to obtain a mixture having gabapentin, wherein the extracting and reacting steps are performed continuously. Preferably, the extracting step is performed using a counter-current extraction method, and more preferably the extracting step is performed using a multistage centrifugal extractor.

In another embodiment, the invention encompasses a method for converting GBP-HCl to gabapentin comprising: providing an aqueous reaction mixture of GBP-HCl; extracting GBP-HCl from the aqueous reaction mixture with an extraction solvent having at least one C₄-C₇ alcohol; reacting the extracted GBP-HCl with a base to obtain a mixture having gabapentin; and recovering crude gabapentin from the mixture by concentrating the mixture to form a slurry, adding a C₃-C₅ alkyl ester to the slurry and recovering the crude gabapentin; and optionally slurrying the crude gabapentin in methanol to provide crystalline gabapentin, wherein the extracting and reacting steps are performed continuously. Preferably, the extracting step is performed by liquid-liquid extraction using a counter-current extraction method, and more preferably the extracting step is performed using a multistage centrifugal extractor.

### DETAILED DESCRIPTION OF THE INVENTION

The present invention provides an efficient method for converting GBP-HCl to gabapentin by liquid-liquid extraction. Isolation and/or purification by conventional methods, such as, continuous separation, precipitation, filtration, washing, and extraction processes are often inefficient and impractical when dealing with large amounts of material such as kilogram quantities. The present invention provides a solution by extracting GBP-HCl and converting this GBP-HCl to gabapentin in kilogram quantities using one solvent throughout which may be suitable for industrial scale synthesis. In addition, the process eliminates the isolation of the intermediate, i.e. GBP-HCl, and allows for solvent recycling. Therefore, the process takes less time, requires fewer steps, is inexpensive to perform and environmentally friendly, and provides gabapentin in high yields. Furthermore, the discarded reaction mixture contains less organic material and therefore requires less environmental treatment.

The present invention encompasses a method for converting GBP-HCl to gabapentin comprising: extracting GBP-HCl from a reaction mixture with an extraction solvent having at least one C₄-C₇ alcohol; and reacting the extracted GBP-HCl with a base to obtain a mixture having gabapentin, wherein the extracting and reacting steps are performed continuously. Preferably, the extraction step is performed by liquid-liquid extraction using a counter-current extraction method, and more preferably the extraction step is performed using a multistage centrifugal extractor. As used herein, unless otherwise defined, the term “performed continuously” refers to carrying out the extracting and reacting steps without isolation of the intermediate GBP-HCl. As used herein, unless otherwise defined, the term “extracting step” refers to extracting GBP-HCl from a reaction mixture with an extraction solvent. As used herein, unless otherwise defined, the term “reacting step” refers to reacting the extracted GBP-HCl with a base to deprotonate GBP-HCl and obtain gabapentin.

Liquid-liquid extraction is a separation technique which involves two immiscible liquid phases having different densities. A quantity of feed liquid may be mixed with a quantity of solvent, after which the layers are settled and separated. In most cases, two liquid phases must be brought in contact to permit transfer of material and subsequent separation of phases. Preferably, the liquid-liquid extraction is performed using an extraction method having counter-current contacts between the two liquid phases. The objective is to strip one or more components from the feed liquid when two liquid phases are passed countercurrent to each other. Counter-current extraction may be operated continuously by supplying a constant flow of feeding solution. The extraction method can be carried out using a variety of devices including, but not limited to, mixer-settlers, pulsating columns, packed columns, and centrifugal extractors. Preferably, the device used in the present invention is a multistage mixer-settler or a multistage centrifugal extractor. More preferably, the device used is a multistage centrifugal extractor. Such devices and methods can be found in HANDBOOK OF SEPARATION TECHNOLOGIES FOR CHEMICAL ENGINEERING section 1.9 “Liquid-Liquid Extraction,” by Lanny A Robbins, Ph.D. (pp 1-255 to 1-339, P. A. Schweitzer ed., McGraw-Hill Book Company, 1979) or UNIT OPERATIONS OF CHEMICAL ENGINEERING Chapter 19, “Leaching and Extraction,” by McCabe et al., (pp 529-558, McGraw-Hill Book Company, 4^{th} ed. 1985), both hereby incorporated by reference.

Multistage centrifugal extraction is one of the most efficient extraction methods because when performing liquid-liquid extraction, it has the ability to include several stages of extraction while having small liquid hold up. The multistage extraction process mixes fluids of two liquid phases thoroughly, extracts the desired substance and separates the two phases efficiently utilizing centrifugal force. Various factors impact the process including, but not limited to, numbers of extraction stages, contact time, feed material and solvent to feed material ratio.

In one embodiment, the invention uses a 4-stage centrifugal extraction system from Rousselet Robatel (Model BXP 130) as the multistage centrifugal extractor. The centrifugal extraction system is available from Robatel, Inc. (Pittsfield, Mass.).

Another method of performing the liquid-liquid extraction step is to use an agitated reactor as disclosed in Examples 4 and 5 of the application. When using an agitated reactor, the reaction mixture (containing GBP-HCl) and extraction solvent are added to a single vessel and contact each other in a cross current manner. Subsequently, the reaction mixture and extraction solvent are mixed by agitation. Devices commonly used for this method can be found in UNIT OPERATIONS OF CHEMICAL ENGINEERING, Chapter 9, “Agitation and Mixing of Liquids” by McCabe, et al., (pp 208-217, McGraw-Hill Book Company, 4^{th} ed. 1985). After agitation, the two phases are allowed to separate and the organic layer is removed from the vessel. Typically, the volume ratio of reaction mixture to extraction solvent is about 3:1. The process may be repeated as necessary such that the aqueous layer may undergo further extraction with additional extraction solvent. The mixing time can be determined by the time sufficient for the mixture of the two phases to reach equilibrium. Settling time can be determined by visual observation of the separation of the phases. Once the extraction has ended, the organic layers are combined and used in the reaction step.

The invention encompasses a method for converting GBP-HCl to gabapentin comprising the steps of: providing an aqueous reaction mixture of GBP-HCl; extracting GBP-HCl from the reaction mixture with an extraction solvent having at least one C₄-C₇ alcohol; reacting the extracted GBP-HCl with a base to obtain a mixture having gabapentin; recovering crude gabapentin from the mixture by concentrating the mixture to form a slurry; adding a C₃-C₅ alkyl ester to the slurry; and recovering crude gabapentin, wherein the extracting and reacting steps are performed continuously. Preferably, the extracting step is performed by liquid-liquid extraction using a counter-current extraction method and more preferably, the extraction step is performed using a multistage centrifugal extractor. Optionally, the process may further comprise slurrying the crude gabapentin in methanol to yield crystallized gabapentin. Slurrying methods that may be used are those described in U.S. Pat. No. 6,255,526, hereby incorporated by reference.

As used herein unless otherwise defined the term “aqueous reaction mixture of GBP-HCl” refers to a solution of gabapentin hydrochloride, water, and solvents suitable for the synthesis of GBP-HCl. The aqueous reaction mixture of GBP-HCl can be prepared according to any method known in the art. For example, the GBP-HCl can be prepared as described in U.S. Pat. No. 6,255,526.

Typically, the aqueous reaction mixture of GBP-HCl is used as the feed solution. The aqueous reaction mixture of GBP-HCl typically contains about 0.1% to about 25% of GBP-HCl by weight. Preferably, the aqueous reaction mixture contains about 3% to about 8% of GBP-HCl. More preferably, about 6% of GBP-HCl is present in the aqueous reaction mixture. Typically, the aqueous reaction mixture has a pH less than about 7. Preferably, the pH is about 2 to about 5. More preferably, the pH is about 3.5 to about 4. The aqueous reaction mixture also contains about 5% to about 20% NaBr and 5% to about 20% NaCl salts. Preferably, the aqueous reaction mixture contains about 15% NaBr and about 13% NaCl.

The extraction solvent is capable of separating impurities, such as separating inorganic salts (NaCl, NaBr) from the GBP-HCl reaction mixture. Typically, the extraction solvent has at least one C₄-C₇ alcohol. The C₄-C₇ alcohol is fed counter currently or bathwise to the extraction system, more preferably, counter currently.

The C₄-C₇ alcohol is preferably selected from the group consisting of n-butanol, iso-butanol, tert-butanol, n-pentanol, hexanol, cyclohexanol, heptanol, amyl alcohol, and a mixture thereof. More preferably, the C₄-C₇ alcohol is iso-butanol and/or amyl alcohol. Most preferably, the C₄-C₇ alcohol is iso-butanol.

Optionally, water is added to the extraction solvent. Dry extraction solvents may be used, however they may absorb water during the synthesis of gabapentin hydrochloride or the extraction process. However, the process of the invention can isolate gabapentin with or without the addition of water in the extraction solvent. Further, it would also be practical to recycle the extraction solvent. Preferably, when water is present in the extraction solvent, then the water added to the extraction solvent is about 0.5% by volume to about the saturation level of the extraction solvent with water. More preferably, the extraction solvent comprises a mixture of about 90% iso-butanol saturated with about 10% water.

The volume ratio of the aqueous reaction mixture to the extraction solvent is about 1:0.5 to about 1:2, respectively. Preferably, the volume ratio is about 1:0.75, respectively.

After the extraction step, the GBP-HCl extract is relatively free of inorganic salts. Preferably, the amount of the inorganic salts remained is at about 10 mg/l to about 120 mg/l. More preferably, the amount of the inorganic salts remained is at about 10 mg/l to about 15 mg/l and most preferably, the amount is less than about 15 mg/l alcohol.

Preferably, the step of reacting GBP-HCl with a base is performed using a steered vessel or a static mixer. More preferably, it is performed using a steered vessel.

During the step of reacting GBP-HCl with a base, the extracted GBP-HCl is heated prior to the addition of a base, preferably, it is heated at a temperature of about 5° C. to about 70° C. More preferably, it is heated at a temperature of about 20° C. to about 45° C. Most preferably, it is heated at a temperature of about 35° C. A base is added to the extract of GBP-HCl in a stoichiometric amount sufficient to form a mixture having gabapentin free base. Preferably, the base is an organic base. More preferably, the base is an amine selected from the group consisting of triethylamine, tributylamine, diisopropylamine, trihexylamine, diethylamine, ethanolamine and benzylamine. Most preferably, the base is tributylamine. Typically, the base is present in a molar ratio of about 1:1 to about 1.5:1 of base to GBP-HCl. Preferably, it is present in a molar ratio of about 1.2:1 base to GBP-HCl.

Gabapentin can be recovered by concentrating the mixture to form a slurry and collecting crude gabapentin from the slurry. The concentrating is achieved by removing the solvent from the mixture, such as by evaporating under elevated temperature and reduced pressure. Preferably, the mixture is fed to an evaporator for concentration and concentration is done continuously with a jacket temperature of about 40° C. to about 140° C. and a typical pressure of about 10 mbar to about 150 mbar. More preferably, the jacket temperature is about 120° C. and the pressure is about 60 mbar. Preferably, the mixture is concentrated to about 8% w/w to about 40% w/w solids. More preferably, it is concentrated to having about 20% w/w to about 30% w/w solids.

Optionally, after the concentration step, a C₃-C₅ alkyl ester is added to the concentrated mixture. Preferably, the C₃-C₅ alkyl ester is selected from the group consisting of methyl acetate, ethyl acetate, propyl acetate and butyl acetate. More preferably, the C₃-C₅ alkyl ester is ethyl acetate. Typically, the C₃-C₅ alkyl ester is present in an amount of about 20% to about 90% by weight of the slurry, and preferably it is present in an amount of about 25% to about 50% by weight of the slurry.

In one example, the crude gabapentin is recovered from the slurry by removing the C₃-C₅ alkyl ester by filtering the slurry in a centrifuge. While the C₃-C₅ alkyl ester is being removed, residues of the C₄-C₇ alcohol, from the extraction step, are also being removed, providing crude GBP. Optionally, these solvents can be recycled.

The crude gabapentin may be committed to a second slurry with the addition of methanol providing crystalline gabapentin form II. Additional details for crystallization of gabapentin are provided in U.S. Pat. No. 6,255,526.

It is readily seen that large amounts of gabapentin can be successfully obtained by liquid-liquid extraction using a counter-current extraction method over other conventional extraction methods.

Having described the invention with reference to certain preferred embodiments, other embodiments will become apparent to one skilled in the art from consideration of the specification. The invention is further defined by reference to the following examples describing in detail the method for converting gabapentin hydrochloride salt to gabapentin by liquid-liquid extraction. It will be apparent to those skilled in the art that many modifications, both to materials and methods, may be practiced without departing from the scope of the invention.

### EXAMPLES

### Example 1

An aqueous reaction mixture of about 6% w/w GBP-HCl, about 13% w/w NaCl and about 15% w/w NaBr at pH of 3.5-4 was used as feed material. The mixture was fed for at least 4 hours at a rate of 120 liters per hour to a 4 stage centrifugal extraction system (Rousselet Robatel BXP 130). The centrifugal extraction system was fed between the 2^{nd} and 3^{rd} stages.

An extraction solvent comprising of a mixture of about 90% w/w iso-butanol saturated with 10% w/w water was fed counter currently to the extraction system at a rate of 120 liters per hour to the 1^{st} stage. Water was fed to stage 4 at a rate of 40 liter per hour. The lean reaction mixture was emitted from stage 1, while the stripped extract was obtained from stage 4.

350 liters of the above mentioned extract (and solvent) was heated to 35° C. in an agitated vessel. 17.5 liters of tributylamine was added during 2 hours. The mixture was maintained at the same temperature for additional 1 hour and then fed to a thin film evaporator for concentration to about 30% w/w solids. Concentration was done continuously with jacket temperature of 120° C. and typical pressure of 60 mbar. The obtained white slurry was accumulated in an agitated vessel. 27 liters of ethyl acetate was added and the mixture was maintained under agitation for 60 minutes. The slurry was filtered in a centrifuge to obtain wet cake of crude gabapentin.

The obtained wet cake was charged to a stirred tank with 2.5 parts of methanol per one part of crude gabapentin. After two hours at 35° C., the slurry was cooled down to 25° C. and filtered on a screening centrifuge. The obtained cake was dried under vacuum until desired dryness. About 15.2 kg (59% yield) of gabapentin form II was obtained. Assay of 99.5% was obtained.

An assay of gabapentin was performed by high performance liquid chromatography (HPLC) under the following conditions: A YMC-ODS-AQ, 5μ, 150×4.6 mm, AQ-302 column was used. The eluent was a mixture of 70% buffer (0.025M of potassium dihydrogen phosphate adjusted to pH 6.0 with 20% KOH) and 30% methanol. The flow rate was 0.5 ml/min, the UV detector was set to 210 mn, and column temperature was at 30° C. The sample volume was 20 μL and the diluent was a mixture of 70% buffer (0.025M of potassium dihydrogen phosphate adjusted to pH 6.0 with 20% KOH) and 30% methanol.

1 mg/ml (accurate concentration) of gabapentin standard in diluent was prepared as the standard solution. 1 mg/ml (accurate concentration) of gabapentin sample in diluent was prepared as the sample solution. Each sample was prepared by the following method: Inject the standard and sample solutions into the chromatography and determine areas of gabapentin using suitable integrator. % gabapentin was calculated using the following equation: % gabapentin=[area gabapentin in sample×concentration of standard×purity of standard]/[area gabapentin in standard×concentration of sample].

### Example 2

An aqueous reaction mixture of about 6% w/w GBP-HCl, about 13% w/w NaCl and about 15% w/w NaBr at pH of 3.5-4 was used as feed material. The mixture was fed for at least 4 hours at a rate of 120 liters per hour to a 4 stage centrifugal extraction system (Rousselet Robatel BXP 130). The centrifugal extraction system was fed between the 2^{nd} and 3^{rd} stages.

An extraction solvent comprising of a mixture of about 90% w/w iso-butanol saturated with 10% w/w water was fed counter currently to the extraction system at a rate of 120 liters per hour to the 1^{st} stage. Water was fed to stage 4 at a rate of 40 liter per hour. The lean reaction mixture was emitted from stage 1, while the stripped extract was obtained from stage 4.

350 liters of the above mentioned extract (and solvent) was heated to 35° C. in an agitated vessel. 17.5 liters of tributylamine was added during 2 hours. The mixture was maintained at the same temperature for additional 1 hour and then fed to a thin film evaporator for concentration to about 20% w/w solids. Concentration was done continuously with jacket temperature of 120° C. and typical pressure of 60 mbar. The obtained white slurry was accumulated in an agitated vessel. The slurry was filtered in a centrifuge to obtain wet cake of crude gabapentin.

The obtained wet cake was charged to a stirred tank with 2.5 parts of methanol per one part of crude gabapentin. After two hours at 35° C., the slurry was cooled down to 25° C. and filtered on a screening centrifuge. The obtained cake was dried under vacuum until desired dryness. About 9.8 kg (38% yield) of gabapentin form II was obtained. Assay of 99.5% was obtained. The assay was performed as described in example 1.

### Example 3

A reaction mixture of aqueous solution of about 6% w/w GBP-HCl, about 20% w/w NaCl and about 20% w/w NaBr at pH of 3.5-4 was used as feed material. The mixture was fed for at least 4 hours at a rate of 1000 ml per hour to a 4 stage laboratory mixer settler extraction system (Rousselet Robatel SX1-1).

An extraction solvent comprising of a mixture of about 90% w/w iso-butanol saturated with 10% w/w water was fed counter currently to the extraction system at a rate of 750 ml per hour to the 1^{st} stage. The lean reaction mixture in water was emitted from stage 1, while the stripped extract in butanol was obtained from stage 4. 1000 ml of the above mentioned organic extract was washed twice with 150 ml of water in a separatory funnel.

The above mentioned organic extract (and solvent) was heated to 35° C. in an agitated vessel. 50 ml of tributylamine was added during 2 hours. The mixture was maintained at the same temperature for additional 1 hour and then fed to a laboratory thin film evaporator for concentration to about 30% w/w solids. Concentration was done with jacket temperature of 120° C. and typical pressure of 60 mbar. The obtained white slurry was accumulated in an agitated vessel. 77 ml of ethyl acetate was added and the mixture was maintained under agitation for 60 minutes. The slurry was filtered to obtain wet cake of crude gabapentin.

The obtained wet cake was charged to a stirred tank with 2.5 parts of methanol per one part of crude gabapentin. After two hours at 35° C. the slurry was cooled down to 25° C. and filtered. The obtained cake was dried under vacuum until desired dryness. About 30 g (44% yield) of gabapentin form II was obtained. The assay of 99.5% was obtained. Assay was performed as described in example 1.

### Example 4

900 ml of an aqueous reaction mixture of 6% GBP-HCl, about 13% NaCl and about 15% NaBr was mixed with 300 ml of amyl alcohol in an agitated 1.5 liter glass reactor. The pH of the combined mixture was adjusted to 3.3 by adding about 2 ml of 32% HCl . After agitated for 15 minutes, agitation was stopped and the organic and aqueous phases were allow to separate. Organic phase was removed and kept for later use and the aqueous phase was returned to the reactor.

Additional fresh 300 ml of amyl alcohol was charged into the agitated reactor. After about 25 minutes the agitation stopped and the phases were allow to separate. Organic phase was removed and kept for later use and the aqueous phase was returned to the reactor.

Additional fresh 300 ml of amyl alcohol was charged into the agitated reactor. After about 15 minutes the agitation stopped and the phases were allow to separate. Organic phase was removed and kept for later use and the aqueous phase was discarded.

All three organic phases were combined and charged to the reactor and 100 ml of water was added to the reactor. The combined mixture was agitated for another 15 minutes and agitation stopped and the phases were allow to separate. The aqueous phase was drained out of the reactor. Additional 100 ml of water was added to the reactor. The mixture was agitated for another 15 minutes and agitation stopped and the phases were allow to separate. The aqueous phase was drained out of the reactor. About 1107 g of organic phase was collected. The organic phase mixture was then heated to 35° C. and 52.3 g of tributylamine (20% molar excess) was added to the mixture in a two hour period.

The amyl alcohol was evaporated until 232 g of thick organic slurry is left in the reactor. Evaporation was done by applying vacuum while keeping the jacket temperature at 35° C. The reactor content was cooled to 20° C. and 60 ml of ethyl acetate was added to the reactor. The slurry was filtered and the cake was washed using 40 ml of ethyl acetate. 45.2 g (90% yield) of crude gabapentin was obtained.

### Example 5

900 ml of an aqueous reaction mixture of 6% GBP-HCl, about 13% NaCl and about 15% NaBr was mixed with 300 ml of isobutyl alcohol in an agitated 1.5 liter glass reactor. The pH of the combined mixture was adjusted to 3.3 by adding about 2 ml of 32% HCl . After agitated for 15 minutes, agitation was stopped and the organic and aqueous phases were allow to separate. Organic phase was removed and kept for later use and the aqueous phase was returned to the reactor.

Additional fresh 300 ml of isobutyl alcohol was charged into the agitated reactor. After about 15 minutes the agitation stopped and the phases were allow to separate. Organic phase was removed and kept for later use and the aqueous phase was returned to the reactor.

Additional fresh 300 ml of isobutyl alcohol was charged into the agitated reactor. After about 15 minutes the agitation stopped and the phases were allow to separate. Organic phase was removed and kept for later use and the aqueous phase was discarded.

All three organic phases were combined and charged to the reactor and 100 ml of water was added to the reactor. The mixture was agitated for another 15 minutes and agitation stopped and the phases were allow to separate. The aqueous phase was drained out of the reactor. Additional 100 ml of water was added to the reactor. The mixture was agitated for another 15 minutes and agitation stopped and the phases were allow to separate. The aqueous phase was drained out of the reactor. About 1259 g of organic phase was collected. The mixture was then heated to 35° C. and 61.1 g (about 20% molar excess) of tributylamine was added to the mixture in a two hour period.

The isobutyl alcohol was evaporated until 279 g of thick organic slurry is left in the reactor. Evaporation was done by applying vacuum while keeping the jacket temperature at 35° C. The reactor content was cooled to 20° C. and 60 ml of ethyl acetate was added. The slurry was filtered and the cake was washed using 40 ml of ethyl acetate. 46.2 g (93% yield) of crude gabapentin was obtained.

We claim:

1. A method for converting gabapentin hydrochloride to gabapentin comprising: extracting gabapentin hydrochloride with an extraction solvent comprising at least one C₄-C₇ alcohol, and reacting the extracted gabapentin hydrochloride with a base to obtain a mixture having gabapentin, wherein the extracting and reacting steps are performed continuously.

2. The method of claim 1, wherein the extraction step is performed by liquid-liquid extraction using a counter-current extraction method.

3. The method of claim 2, wherein the counter-current extraction method comprises feeding a solution of gabapentin hydrochloride in an aqueous solvent into a first extraction unit and feeding an organic solvent having a different density than the aqueous solvent and being immiscible with the aqueous solvent into a second extraction unit, wherein each unit is in fluid communication with at least two other immediate units in such way to allow for a one way flow of the solvents in counter-current fashion from one unit to another.

4. The method of claim 2, wherein the counter-current extraction is performed in a mixer-settler, a multistage centrifugal extractor, a pulsating column or a packed column.

5. The method of claim 4, wherein the counter-current extraction is performed in a multistage centrifugal extractor.

6. The method of claim 1, wherein the extracting step is performed by liquid-liquid extraction using a cross-current extraction method.

7. The method of claim 6, wherein the cross-current extraction method comprises a) adding an aqueous reaction mixture of gabapentin hydrochloride and an extraction solvent to a single vessel, wherein the aqueous reaction mixture and the extraction solvent contact each other in a cross current manner; b) mixing the aqueous reaction mixture and the extraction solvent by agitation providing two phases; c) separating the two phases; and d) optionally repeating steps a) through c).

8. The method of claim 6, wherein the cross-current extraction is performed in an agitated reactor.

9. The method of claim 1, wherein the gabapentin hydrochloride is in an aqueous reaction mixture.

10. The method of claim 9, wherein the aqueous reaction mixture contains about 0.1% to about 25% of gabapentin hydrochloride and has a pH less than about 7.

11. The method of claim 10, wherein the aqueous reaction mixture contains about 3% to about 8% of gabapentin hydrochloride and has a pH of about 3 to about 5.

12. The method of claim 1, wherein the C₄-C₇ alcohol is selected from the group consisting of n-butanol, iso-butanol, tert-butanol, n-pentanol, hexanol, cyclohexanol, heptanol, amyl alcohol, and a mixture thereof.

13. The method of claim 12, wherein the C₄-C₇ alcohol is iso-butanol or amyl alcohol.

14. The method of claim 1, further comprising mixing water with the extraction solvent.

15. The method of claim 1, wherein the base is an amine selected from the group consisting of triethylamine, tributylamine, diisopropylamine, trihexylamine, diethylamine, ethanolamine and benzylamine.

16. The method of claim 15, wherein the base is tributylamine.

17. The method of claim 1, wherein the base is present in a molar ratio of about 1:1 to about 1.5:1 of base to gabapentin hydrochloride.

18. The method of claim 17, wherein the base is present in a molar ratio of about 1.2:1 of base to gabapentin hydrochloride.

19. The method of claim 9, wherein the volume ratio of the aqueous reaction mixture of gabapentin hydrochloride to the extraction solvent is about 1:0.5 to about 1:2.

20. The method of claim 19, wherein the volume ratio of the aqueous reaction mixture of gabapentin hydrochloride to extraction solvent is about 1:0.75.

21. The method of claim 1, wherein the extracted gabapentin hydrochloride contains about 10 mg of inorganic salts per one liter of the extraction solvent to about 120 mg of inorganic salts per one liter of the extraction solvent.

22. The method of claim 21, wherein the extracted gabapentin hydrochloride contains about 10 mg of inorganic salts per one liter of the extraction solvent to about 15 mg of inorganic salts per one liter of the extraction solvent.

23. The method of claim 1, further comprising recovering crude gabapentin from the mixture by concentrating the mixture to form a slurry; adding a C₃-C₅ alkyl ester to the slurry and recovering crude gabapentin from the slurry; and optionally slurrying crude gabapentin with methanol to provide crystalline gabapentin.

24. The method of claim 23, wherein the crystalline gabapentin is crystalline gabapentin Form II.

25. The method of claim 23, wherein the C₃-C₅ alkyl ester is selected from the group consisting of methyl acetate, ethyl acetate, propyl acetate and butyl acetate.

26. The method of claim 25, wherein the C₃-C₅ alkyl ester is ethyl acetate.

27. A method for converting gabapentin hydrochloride to gabapentin comprising: extracting gabapentin hydrochloride with an extraction solvent comprising at least one C₄-C₇ alcohol, and reacting the extracted gabapentin hydrochloride with a base to obtain a mixture having gabapentin, wherein the extraction step is performed by liquid-liquid extraction using a counter-current multistage centrifugal extractor, and the extracting and reacting steps are performed continuously.
